(19) United States
(12) Patent Application Publication
(10) Pub. No.: US 2022/0088278 A1
(43) Pub. Date: Mar. 24, 2022

(54) Covered Stent For Local Drug Delivery

(71) Applicant: BIOTRONIK AG, Buelach (CH)

(72) Inventors: Ronald E. Betts, La Jolla, CA (US); Douglas R. Savage, La Jolla, CA (US)

(21) Appl. No.: 17/423,669

(22) PCT Filed: Jan. 28, 2020
(86) PCT No.: PCT/EP2020/052005

§ 371 Date: Jul. 16, 2021

Related U.S. Application Data
(60) Provisional application No. 62/798,498, filed on Jan. 30, 2019.

(51) Int. Cl. A61L 31/16; A61L 31/02; A61L 31/10; A61L 31/14; D04H 1/728; D04H 1/435; A61K 31/436

(52) U.S. Cl. CPC A61L 31/16; A61L 31/022; A61L 31/10; A61L 31/146; A61L 2420/02; D04H 1/435; A61K 31/436; A61L 2300/416; D04H 1/728

(57) ABSTRACT

The present invention relates generally to an endoprosthesis for maintaining patency of a body vessel, e.g., a stent, in a basically tubular configuration comprised of a structural lattice with a mesh covering which is capable of storing releasing one or more drugs to and penetrating into surrounding tissue.

### FIELD OF THE INVENTION

The present invention relates generally to an endoprosthesis for maintaining patency of a body vessel, e.g., a stent, in a basically tubular configuration comprised of a structural lattice with a mesh covering which is capable of storing releasing one or more drugs to and penetrating into surrounding tissue.

### BACKGROUND OF THE INVENTION

Stents are tubular devices made of plastic or metal and are used to develop or maintain patency of an animal or human vessel. Stents may be placed endoscopically or through percutaneous methods to treat the common bile duct or the main pancreatic duct to relieve obstruction. These devices effectively are employed to drain gallbladder and pancreatic fluid collections. Plastic stents represent the lower cost alternative of the devices available but they are limited in diameter to 12 French (0.156 inches) which is the allowable maximum possible that can be accommodated by a standard endoscope. For drainage of larger vessels several plastic stents or an expandable metal stent would be used. Metallic stents can be balloon expandable or constructed of materials which are self-expanding upon deployment from a delivery catheter. Either type of metallic stent can be made available with a covering made of polymer or silicone. This covering prevents ingress of tissue from the surrounding vessel lumen.

Obstruction of the bile duct or pancreatic duct can occur due to benign hyperplasia resulting from inflammation or from malignancy of the pancreas or bile duct and surrounding tissues. Its development may contribute to poor clinical outcomes including pain and delay in further treatment. Biliary obstruction correlates with decreased survival times in cancer patients. In the U.S. over 50,000 persons and over 400,000 persons worldwide are diagnosed with pancreatic cancer each year and as many as 70% of patients have some degree of biliary obstruction at the time of diagnosis. Pancreatitis affects 9 million persons worldwide. The role of biliary stents in achieving patency in obstructions or strictures of the bile duct has been well established with more recent studies showing an increased role for self-expanding metal stents. An increasing number of patients with resectable, or surgically treatable pancreatic cancers are receiving neoadjuvant chemoradiation or chemotherapy prior to surgery to increase its potential benefit. With a neoadjuvant approach surgery is delayed many months and hence there is a need for effective and durable biliary drainage, particularly driven by the fact that many chemotherapy agents require adequate liver function.

Maintaining patency of the bile duct with a stent is a requirement for further treatment of disease and to facilitate surgery at a later date. Yet review of the available literature reveals that the failure rate of stents used for treatment of malignant obstruction ranges from 19% to 46%. The predominant cause of stent failure is hyperplasia or tumor growth either into or around the implanted stent. Clogging of the stent lumen with bile salts or impacted food constitutes the secondary cause of stent failure. The addition of a covering material to the stent will reduce but not eliminate tissue ingrowth. The surface of the prosthesis may be covered entirely or it may be constructed so that defined ends of the device are exposed without covering.

Previous attempts have been made to release drug from a covered prosthesis. A membrane comprised of polymer and the drug paclitaxel was wrapped around a conventional self-expanding stent and deployed in the main bile duct of thirty-six pancreatic cancer patients in an effort to maintain patency of the duct during neoadjuvant therapy or while waiting for surgery. The duct patency duration of the patients did not significantly differ from those patients who had traditional treatment with a covered stent without drug. Several possible explanations could explain this outcome. The paclitaxel drug may not have been stable or effective in the higher pH environment present in the main bile duct. The amount of drug or the availability of the drug to surrounding tissue may have been inadequate. The physical configuration of the stent used in the study could have been prone to blockage from food or bile contents. The intent of the present invention is to overcome both possible shortcomings of the previously studied device.

It is therefore the object of the present invention to provide for a drug eluting stent system for drug delivery in the bile or pancreatic duct which is capable providing a suitable therapeutic agent in an effective dose to prevent ingress or overgrowth of hyperplasia and tumor materials.

### SUMMARY OF THE INVENTION

The present invention provides for a drug eluting stent system comprising a coating layer, wherein the coating is composed of a porous surface covering and at least one therapeutic agent comprising an antiproliferative agent, preferably a macrocyclic triene immunosuppressive compound. The coating layer is applied to a stent being capable of radial expansion in manner that the stent is encapsulated by the coating.

In one aspect it is provided a drug eluting stent system for use in delivering at least one therapeutic agent to a target, preferably the bile or pancreatic duct, comprising:
(a) a stent capable of radial expansion; and
(b) a coating, wherein the coating is composed of a porous surface covering and at least one therapeutic agent, wherein the at least one therapeutic agent is an antiproliferative agent and wherein the stent is encapsulated by the coating.

The porous surface covering can be configured in the form of a mesh and would ideally be biocompatible with surrounding tissue and would maintain the flexibility and radial strength of original uncovered stent. At least one portion of the porous surface covering has sufficient porosity to contain enough therapeutic agent to provide therapeutic benefit over the full period of treatment. The porous surface covering is provided with an interior surface at the luminal side of the drug eluting stent system and an exterior surface at the abluminal side of the drug eluting stent system. Both, the interior and the exterior surface can have the same properties and belong to a surface covering of the same material. The drug eluting stent system can also be provided with interior and exterior surfaces of the surface covering having different properties, in particular if the surface covering encapsulating the stent is produced of two different sheets of a porous material.

The drug eluting stent system is preferably provided with a smooth contiguous interior surface to prevent compaction of fluid and food materials and also an effective means to prevent ingress or overgrowth of hyperplasia and tumor materials. The morphology of the interior of the stent surface may be of lesser porosity than the exterior surface. The drug eluting stent system delivers drug over prescribed time duration while maintaining the patency of the vessel and minimize or inhibit tissue ingrowth.

A drug which is to be administered in the bile duct or pancreatic duct is required to have a superior efficacy and stability. The drug is desired to have both anti-proliferative and anti-inflammatory properties to enable suppression tumor and endothelial growth. In the living tissue the drug would inhibit benign or malignant hyperplasia in the particular vessel environment where deployed. The drug should be non-covalently bound to the porous surface covering in order to deliver a measured release of the drug at a target site. Particular consideration is given to the pH of the bile duct fluid which differs from that which is present in venous or arterial blood. Hence, the drug needs to be stable in basic pH environment. It is also desirable that the drug be lipid soluble to facilitate absorption into the surrounding tissue. It is further desirable that the drug have a wide dosing window of therapy. Specifically, the drug should exert a therapeutic effect over a range of low to high dosage levels without concern for local toxicity. It is possible that the lack of efficacy of biliary stents with embedded paclitaxel may have been due to low compatibility for one or more of the attributes stated above.

Hence, in one aspect, the present invention provides for a drug eluting stent system comprising a macrocyclic triene immunosuppressive compound which meets all the above requirements and has the following structure:

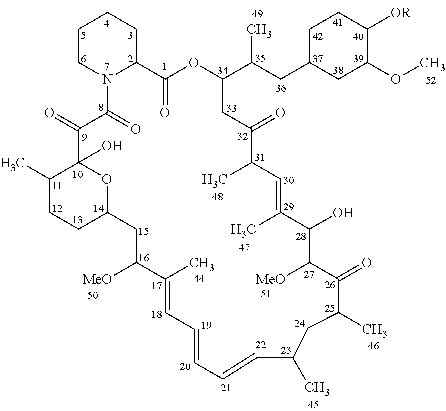

where R is C(O)—(CH₂)ₙ—X, n is 0, 1 or 2, X is a cyclic hydrocarbon having 3-9 carbons and optionally contains one or more unsaturated bonds as a coating material for medical implants, in particular for vascular stents. In a preferred embodiment of the use of the mixture, C(O)—(CH₂)ₙ—X has one of the following structures:

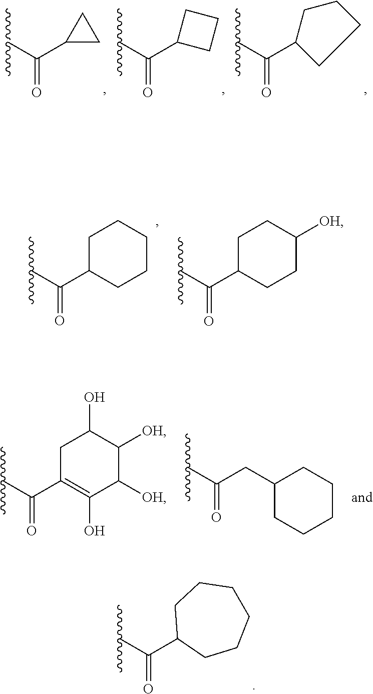

In another aspect the present invention is directed to a method for preparing a drug eluting stent system comprising the steps of
(a) preparing a sheet of a porous material on a surface,
(b) mounting the sheet on at least the luminal or abluminal surface of a stent,
(c) applying elevated temperature and pressure to encapsulated the stent in the porous material thereby providing a porous surface covering on the stent,
(d) applying a therapeutic drug formulation to the porous surface covering, and
(e) drying the porous surface covering applied with the therapeutic drug formulation.

The therapeutic drug formulation applied to the covered stent must meet the requirements for manufacturability and efficacy. The therapeutic drug formulation comprises at least one therapeutic agent as defined herein and must be dissolved into a suitable solvent and applied as a liquid formulation through spraying, capillary application, or dip coating. The drug formulation would be maintained within porous surface covering (e.g. in the mesh lattice) as a solid mass or as captive microparticles or microspheres. It is possible that selective areas of the stent geometry could also be coated with the drug if desired. Once dried the drug must be stable at room temperature over the usable lifetime of the product. The therapeutic drug formulation comprising at least one therapeutic drug may be applied to the inner surface, the outer surface, or to both inner and outer surfaces.

### BRIEF DESCRIPTION OF THE FIGURES

FIG. 1 is a photograph of a self-expanding stent which is encapsulated with two layers of porous electrospray PTFE film.

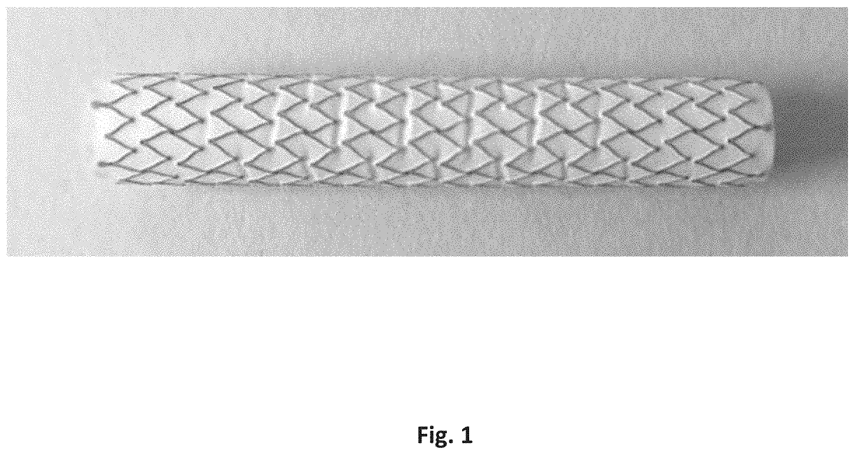

FIG. 2 is a close-up image of the encapsulated stent showing film coverage over the strut.

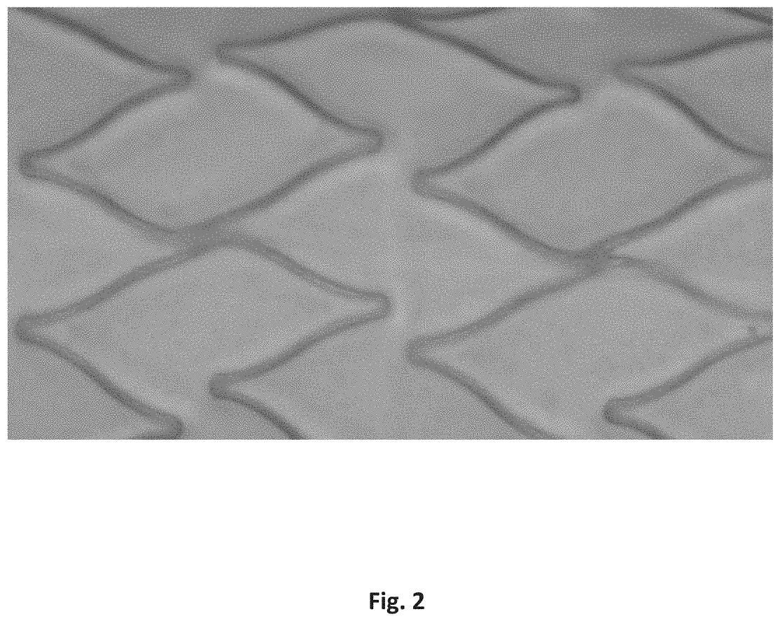

FIG. 3 is a scanning electron microscope image of the cross-section of the encapsulated device showing the stent struts surrounded by the porous mesh material.

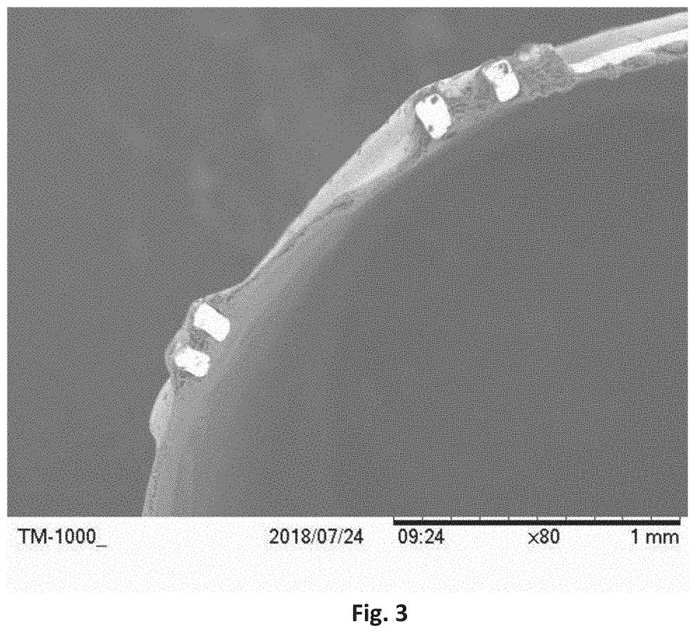

FIG. 4 is a scanning electron microscope image of the cross-section of the encapsulated device showing a single stent strut surrounded by the porous mesh material.

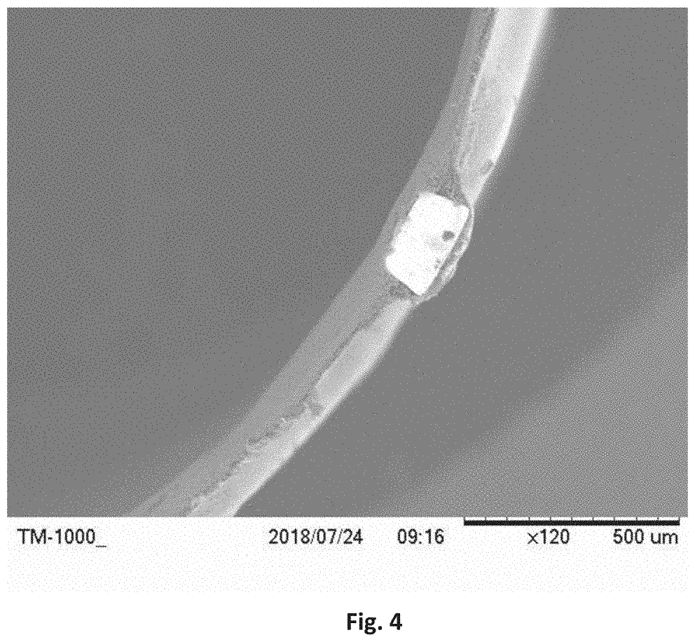

FIG. 5 is a scanning electron microscope image of the porous mesh material as it appears in a cut cross-section.

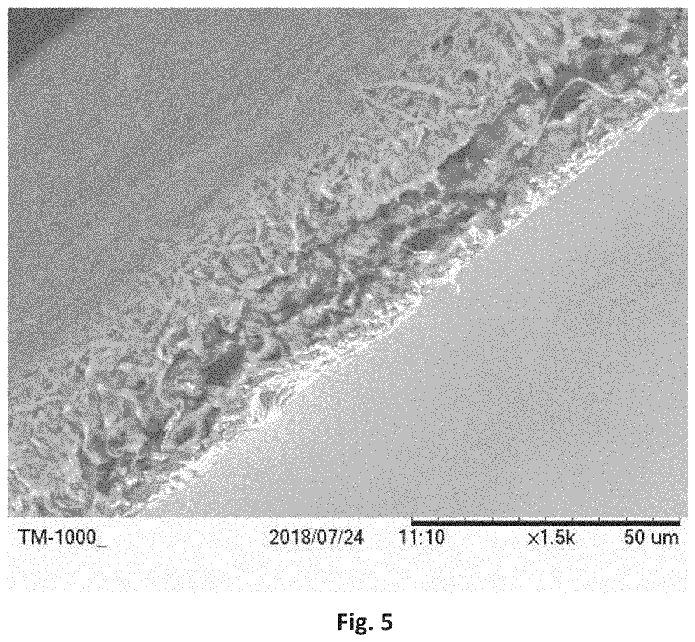

FIG. 6 is a scanning electron microscope image of the top view of the porous mesh with dimensions indicating the approximate pore diameter of 2.0 to 5.0 microns.

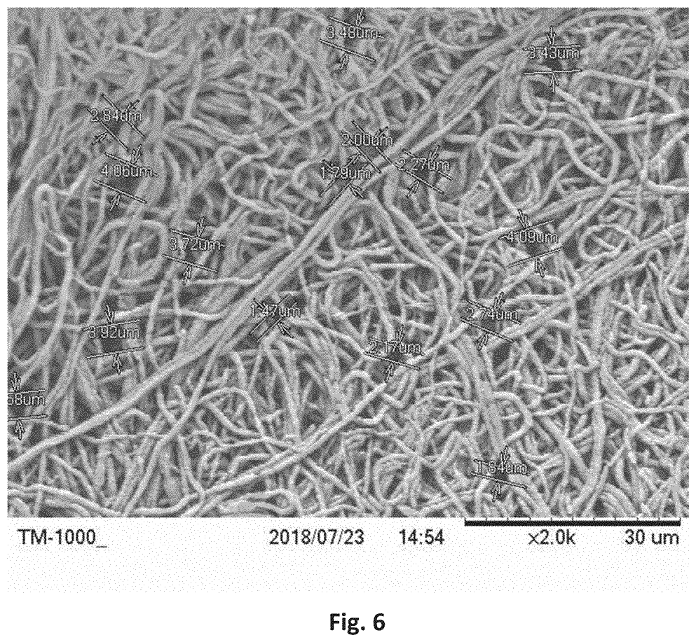

FIG. 7 is a scanning electron microscope image of the top view of the porous mesh at high magnification.

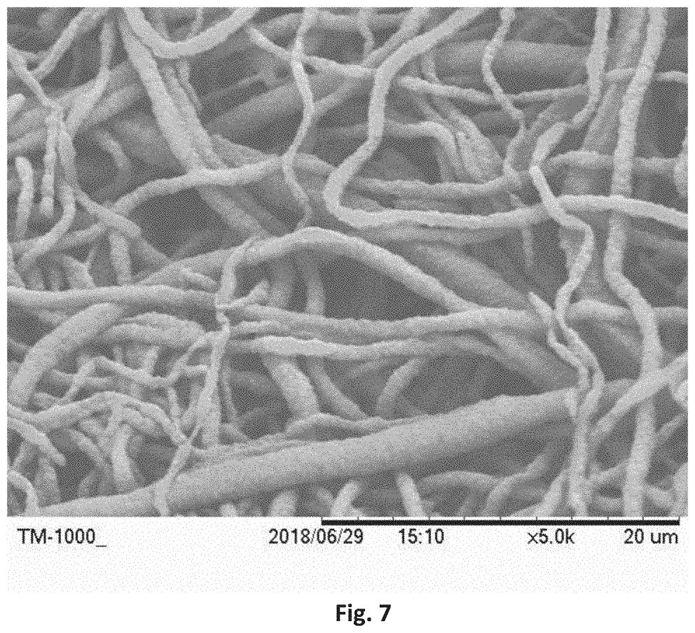

FIG. 8 is a scanning electron microscope image of the top view of the porous mesh with drug formulation applied into the mesh surface.

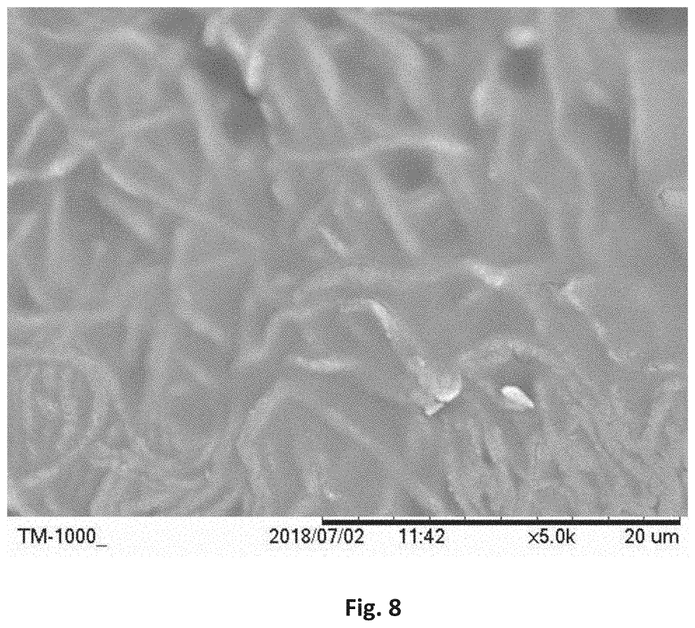

FIG. 9 is a rendering of cross-sectional view of two design configurations consisting of a two-sheet encapsulation of PTFE and a single electrospray application to the stent.

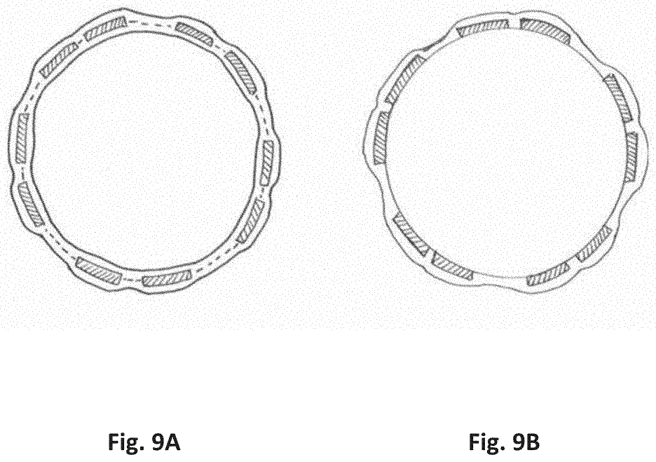

FIG. 10 is graph of incremental measured amount over time of CRC-015 drug released from the drug coated device in an appropriate liquid media.

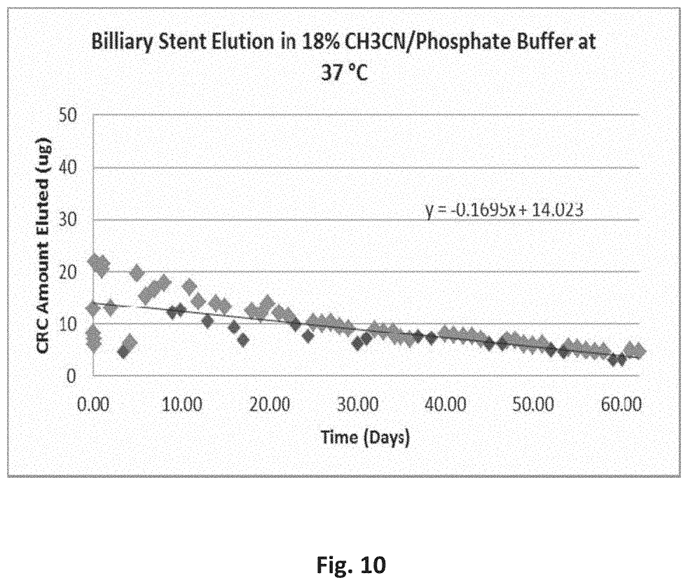

### DETAILED DESCRIPTION OF THE INVENTION

### Definitions

As used herein, the term “coated” or “coating” with reference to a stent refers to coverings, compounds or substances applied to the stent surface. This would also include a drug or therapeutic compound in or on a covering, as well as any other substance that is applied to the stent surface in order to facilitate the delivery of the drug from the stent surface to a target.

As used herein, the term “elution” refers to the transfer of a drug or therapeutic agent out of the coating and is determined as the total amount of the drug or therapeutic agent excreted out of the porous surface covering as integral part of the coating.

As used herein, the term “polymer” refers to substances composed entirely of repeating structural units or monomers, connected to each other via covalent chemical bonds.

As used herein, the term “subject” refers to any animal, including humans, for which the administration of an adjuvant composition is desired. It includes mammals and non-mammals, including primates, livestock, companion animals, laboratory test animals, captive wild animals, reptiles, and fish.

As used herein, the term “encapsulated” refers to a complete surrounding of a material element with another material.

### Stent

The stent of the present invention may be produced from a suitable biocompatible metal and capable of insertion into the vasculature of an individual. The stent is comprised of a metallic base comprising a material such as stainless steel, titanium or a similar biocompatible alloy thereof.

The stent of the present invention is substantially tubular or cylindrical in design and is capable of radial expansion when located at a desired target, such as within the vasculature or proximal to a vessel wall. The stent can be configured to be self-expanding or balloon expandable.

### Coating

The present invention provides for a coating which comprises a porous surface covering that is specifically constituted to carry an extraordinarily large drug load. The porous surface covering encapsulates the stent. It is in the sense of the present application that the coating in the form of a porous surface covering expands over the interstices between stent struts as the porous surface covering formed from porous sheets which are places on the interior and/or exterior surface of the stent. The encapsulation is accomplished by applying elevated temperature and pressure to at least one sheet of porous material mounted on the stent. The porous material is in one embodiment a nonwoven fabric. The nonwoven fabric is constituted of polymeric fibers. Temperature and pressure are chosen to transfer the polymer into a semi molten state such that the fibers stick to one another upon compression. As a result of the compression a porous stent covering in the form of a thin web or mesh is produced capable of maintaining flexibility and structural integrity and further of loading and storing greater quantities of a therapeutic agent as described herein. Also, after the application of elevated temperature and pressure the porous surface covering has a sponge-like configuration capable of taking up large amounts of a drug containing formulation. The fibers of the nonwoven fabric preferably have an average diameter of 0.25 to 2.00 micrometer. Variation of the diameter of the fibers can be used to adopt a specifically desired porosity. With smaller diameters of the fibers lower degrees of porosity can be achieved. In such an embodiment the diameters of the fibers can range from 0.25 to 0.85 micrometer. Also, the interior and the exterior surface of the porous covering can have the same properties such as the porosity and belong to a surface covering of the same material. Further, the porous covering can be provided with interior and exterior surfaces having different properties. Variations in properties can reside in different porosities, materials, hydro- or lipophilicity, densities or the like as well as combinations of the aforementioned. The luminal (interior) surface is desired to be smooth in order to reduce entrapment of biliary sludge and food. In such embodiment the interior surface of the porous covering exhibits a low degree of porosity, preferably below 5%, or no porosity at all. No porosity at all could be accomplished by providing a plain polymer layer e.g. in the form of a polymer film. The abluminal surface should be of sufficient porosity to store elevated therapeutic levels of drug and to efficiently elute the drug from the stent system. In that, a porous surface covering of the drug eluting stent system is suggested exhibiting a porosity gradient increasing from the luminal surface to the abluminal surface of the stent system. In one embodiment of the drug eluting stent system the porous covering has a gradient of porosity from 0% to 5% porosity at the interior (luminal) surface to 20 to 40% porosity at the exterior (abluminal) surface. Also, one embodiment of the drug eluting stent system the porous covering has a gradient of density from a high density at the interior (luminal) surface to a lower density at the exterior (abluminal) surface. The highest density of the porous covering is that of the plain polymeric material used for fibers without exhibiting any porosity at all. It is in the sense of the invention described herein that one surface side (interior or exterior) may exhibit no porosity at all simultaneously requiring that the other side exhibits porosity to be a porous surface covering. In that at least one surface exhibits porosity, preferably the external (abluminal) surface. It shall be understood that “surface side” is directed to the surface sides of the stent having an interior, luminal side and an exterior, abluminal side. Having a surface covering from one surface side to the other necessarily leads to an encapsulation of the stent since both surfaces of the stent are covered. The thickness of the entire porous surface covering preferably ranges between 20 to 100 micrometers. In this regime the porous surface covering together with the stent is flexible and yet strong enough to resist the external pressure from tissue ingrowth.

In one aspect, the amount of the at least one therapeutic agent applied to the drug eluting stent system is between 0.5 μg/mm² to 5.0 μg/mm² of surface area, preferably from about 1.0 μg/mm² to 3.0 μg/mm² of surface area, depending on stent size. In another preferred embodiment, the drug load of the at least one therapeutic agent per unit area of the stent is from about 4.0 μg/mm² to 5.0 μg/mm² of surface area. Note that this is the plain area without taking the area of the pores into account. Hence, the at least one therapeutic agent as defined herein is present in the porous surface covering in a concentration of at least 0.05 μg/mm², preferably of at least 0.5 μg/mm², more preferably of at least 1.0 μg/mm², and most preferably of at least 3.0 μg/mm² of surface area. Further in a specific embodiment, the amount of the at least one therapeutic agent between 0.5 μg/mm² to 5.0 μg/mm² of surface area, preferably between 4.0 μg/mm² and 5.0 μg/mm² is applied only to the abluminal side to the drug eluting stent system. Hence, only the abluminal surface of the porous covering is exhibiting these drug loads, whereas the luminal surface of the porous stent covering is free of the at least one therapeutic agent or exhibits only a drug load of 0.05 μg/mm² to 1.0 μg/mm².

In another aspect, the therapeutic agent is preferably applied to the porous surface covering by use of a formulation comprising at least 2.0% by weight of the at least one therapeutic agent dissolved into a suitable solvent. For preparation of the formulation a solvents are preferred that are nontoxic, capable of dissolving the therapeutic agent and have a vapor pressure low enough to facilitate easy drying. Examples for solvents that fulfill at least one of these requirements are acetone, acetonitrile, tetrahydrofuran, chloroform, dichloromethane, and ethanol.

In a preferred embodiment the porous surface covering is a nonwoven fabric. The nonwoven fabric is preferably produced by way of electrospinning. During electrospinning, a polymer solution, preferably a solution comprising a nonwoven polymer as listed above, is delivered through a metal nozzle. Between the metal nozzle and the basic structure, a high voltage is applied. The basic structure has a different potential than the metal nozzle. By feeding the polymer solution and due to the voltage difference between the nozzle and the basic structure, a filament is applied onto a surface as a nonwoven fabric. The fibers of the nonwoven fabric are composed of a biocompatible polymer and preferably of a biostable biocompatible polymer. In one embodiment of the invention the electrospun fibers are composed of a polymer selected from the group comprising or consisting of include polytetrafluoroethylene, fluorinated ethylene propylene, Dacron, polyethylene terephthalate, polyurethanes, polycarbonate, polypropylene, Pebax, polyethylene and biological polymers such as collagen, fibrin, and elastin. In a further preferred embodiment the electrospun fibers are composed of polytetrafluoroethylene or polyurethane. Further, for encapsulating the stent in the porous covering, two different sheets of non-woven fabric can be applied to the luminal side of the stent and the abluminal side of the stent, respectively. In a preferred embodiment the interior surface of the porous covering is produced of polytetrafluoroethylene with low, preferably below 5%, or no porosity. Such an interior surface of the porous covering could be made of a polytetrafluoroethylene sheet as a material, preferably with a smaller fiber diameter as 1 μm, adds additional strength to the covering, which prevents ingress of hyperplasia and tumor materials. Also, additional to the encapsulation and the application of a therapeutic agent, at least one of the porous and preferably non-woven sheets used for encapsulation of the stent can carry a sealing agent to reduce or eliminate porosity at one surface, preferably the luminal surface, of the porous covering. Suitable sealing agents include but are not limited to silicone, silane compounds, polyurethanes, polysulfones, thermoplastic elastomers, pvc and other vinyl materials, fatty alcohols including docosanol, polysorbate 80 and other surfactants, and flexible, biocompatible organic polymers such as polycaprolactone.

The porous surface covering encapsulating the stent is preferably configured to have a mesh pore diameter ranging from one micron to five microns. Determination of the mesh pore diameter may done under scanning electron microscope inspection. In one aspect of the invention the porous surface covering has a porosity of within the range of 20% to 40% of the surface area and preferably of 30%. As stated above porous surface covering encapsulating the stent may have a gradient of porosity, preferably increasing in porosity from the luminal surface to the abluminal surface of the porous surface covering.

### Therapeutic Agents

The at least one therapeutic agent is a macrocyclic triene immunosuppressive compound having the following structure:

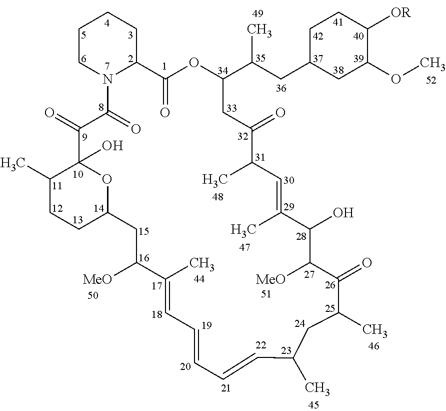

where R is C(O)—(CH₂)ₙ—X, n is 0, 1 or 2, X is a cyclic hydrocarbon having 3-9 carbons and optionally contains one or more unsaturated bonds. In a most preferred embodiment, C(O)—(CH₂)ₙ—X has one of the following structures:

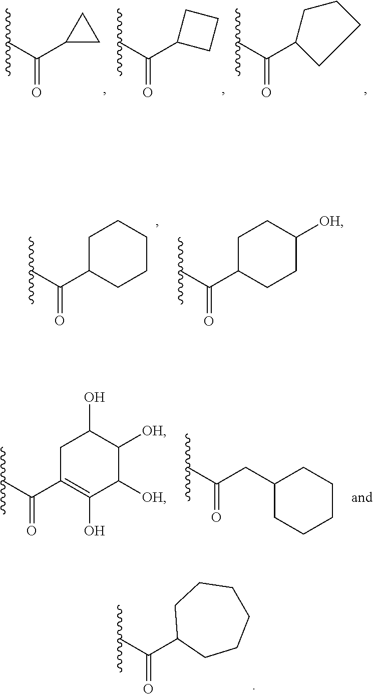

Specifically, the macrocyclic triene immunosuppressive compound of the present invention has more than one embodiment and may be described as comprising at least one of the following species from Table 1:

| | R is C(O)—(CH₂)ₙ— | |
|---|---|---|
| | X having one of | |
| | the following | |
| Main structure | structures | Species |
| 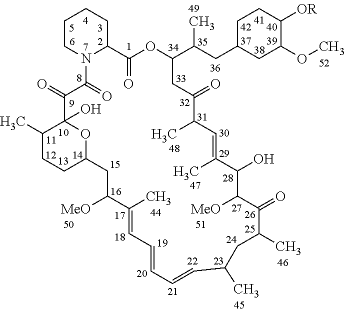 | 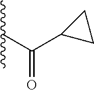 [SMILES: *C(=O)C1CC1] 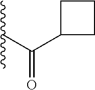 [SMILES: *C(=O)C1CCC1] 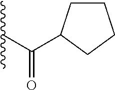 [SMILES: *C(=O)C1CCCC1] | CRC-015a CRC-015b CRC-015c |
| | 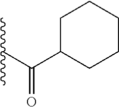 [SMILES: *C(=O)C1CCCCC1] | CRC-015d |
| | 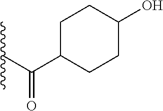 [SMILES: *C(=O)C1CCC(O)CC1] | CRC-015e |
| | 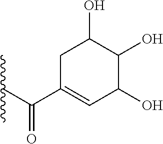 [SMILES: *C(=O)C1=CC(O)C(O)C(O)C1] | CRC-015f |
| | 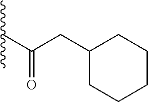 [SMILES: *C(=O)CC1CCCCC1] | CRC-015g |
| | 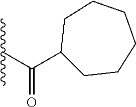 [SMILES: *C(=O)C1CCCCCC1] | CRC-015h |

CRC-015 is a term meant to encompass a genus and used to refer to each of the following species from Table 3: CRC-015a, CRC-015b, CRC-015c, CRC-015d, CRC-015e, CRC-015f, CRC-015g and CRC-015h.

A combination of several drugs may be applied to the stent system surface or stent system surfaces. Among the macrocyclic triene family of compounds besides the CRC-15 compounds are the drugs everolimus, zotarolimus, Biolimus, sirolimus, and Novolimus. Other drugs used to systemically treat pancreatic adenocarcinoma may be applied locally through the use of the implantable stent graft. These drugs could include gemcitibane, paclitaxel, nab-paclitaxel, docetaxel 5-fluorouracil (FU), flurouracil, irinotecan, oxaliplatin, cic-platinen, and leucovorin.

Further, it is recognized that drug combinations can have increase therapeutic effect. The CRC-015 compounds disclosed herein can be combined with cancer drugs below in conjunction with the covered stent of the present invention. These cancer drugs can be selected from the group comprising or consisting of Capecitabine, Erlotinib, Fluorouracil (5-FU), Gemcitabine, Irinotecan, Leucovorin, Paclitaxel, Nab-paclitaxel, Irinotecan and Oxaliplatin.

In one embodiment of the drug eluting stent system the surface covering has different therapeutic agents loaded to the luminal surface and the abluminal surface. One both surfaces different combinations of drugs can be loaded. In one embodiment on the abluminal surface at least one or the only therapeutic agent is a macrocyclic triene immunosuppressive compound having the following structure:

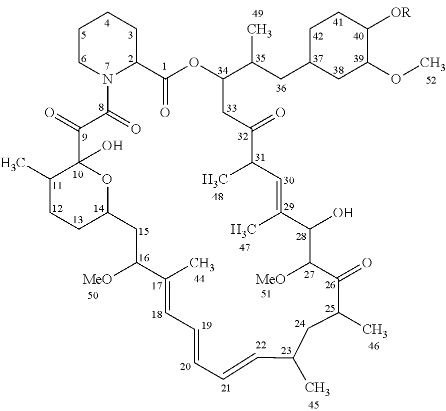

where R is C(O)—(CH₂)ₙ—X, n is 0, 1 or 2, X is a cyclic hydrocarbon having 3-9 carbons and optionally contains one or more unsaturated bonds. In a most preferred embodiment, C(O)—(CH₂)ₙ—X has one of the following structures:

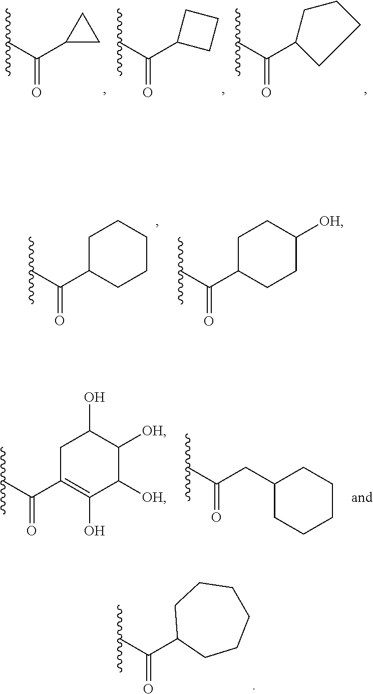

In a further embodiment the drug eluting stent system has loaded at least one of the CRC-15 compounds as defined herein one the abluminal surface and a suitable anti-coagulant on the luminal surface such as heparin. Antibiotics and other drugs or antimicrobial materials which inhibit bacterial film formation may also be applied to the luminal surface.

The at least one therapeutic agent of the coating of the drug eluting stent system as suggested herein may be accompanied by at least one suitable excipient material. Suitable excipient materials may have an effect to prolong the presence of the drug in tissue and are selected from the group comprising or consisting of polyester polymers such as polylactic acid (PLA) and/or polyglycolic acid (PGA), fatty alcohols, fatty acids, lipids, steroids, polymers, waxes, and oils.

Elution

The drug eluting stent of the present invention further provides for a measured elution rate based on the formulation and the nature of the therapeutic agent applied to the porous surface covering and the porosity of the latter. The coating provides for a stable elution rate that is calculated based on the disassociation of the drug from the covering once the stent system is deployed. It is an advantage of the present invention that the porous surface covering is capable of storing higher drug doses that are prerequisite to cancer therapy.

In one aspect the present invention is therefore directed to a method of treating (i) bile duct obstruction or (ii) pancreatic duct obstruction or (iii) pancreatic cancer by placing a drug eluting stent system as described herein into the bile duct or the pancreatic duct.

### Method

One aspect of the invention is directed to a method for preparing drug eluting stent system comprising the steps of
(a) preparing at least one sheet of a porous material on a surface,
(b) mounting the at least one sheet on at least the luminal or abluminal surface of a stent,
(c) applying elevated temperature and pressure to encapsulated the stent in the porous material thereby providing a porous surface covering on the stent,
(d) applying a solution of a therapeutic agent to the porous surface covering, and
(e) drying the porous surface covering applied with the therapeutic agent solution.

In one embodiment step (c) is conducted in such a manner that the temperature is in the range of 55° C. and 90° C. and the pressure is in the range of 0.13 MPa to 1.25 MPa. Further, step (b) may be conduct in such a manner that the sheet is mounted on the luminal and abluminal surface of a stent. Also, the method as suggested herein can be carried out such that steps (a) and (b) are conducted simultaneously by applying the sheet of porous material directly on the surface of the stent. Step (a) is preferably carried out by electrospray technique or other means, especially electro-spinning technique onto a flat surface. Respective sheets are then cut to size before application to the preferably metallic bare stent. Preferably step (a) is conducted in such a way that two sheets of porous material, preferably of electrospun material are provided. Further, step (a) is conducted in such a way that two different sheets of porous material, preferably of electrospun materials are provided. Step (b) is then correspondingly carried out that one of the two sheets, of the same or of a different material, is mounted on the luminal side of a stent, whereas the second sheet of porous material is mounted on the abluminal side of the stent. Also, when a porosity gradient shall be provided for the drug eluting stent system as suggested herein more than one sheet having a different degree of porosity may be provided on each side of the stent in order to provide a more diverse gradient. In particular, it is preferred that the porosity increases from the luminal surface to the abluminal surface of the stent system. In particular for providing the interior surface of the porous covering smooth and with low or no porosity step (a) can be carried out in that at least one sheet of porous material is provided and a second sheet with less or no porosity is provided. In this embodiment the sheet of porous material is mounted on the abluminal surface of the stent, whereas the sheet of less or no porosity is mounted on the luminal surface of the stent. The resulting encapsulation of the stent after carrying out step (c) would then be accomplished between one porous material on the abluminal surface and one non-porous material on the luminal surface. Further, when a porous material on the abluminal surface is combined with a material of less porosity on the luminal surface, step (c) can be carried out in such a way that the few and/or tight pores at the luminal surface are closed by using elevated temperatures, in particular by slightly melting the material at luminal surface. Additionally, step (f) may be incorporated in the method as suggested herein comprising applying at least one sealing agent to the luminal surface of the porous surface covering.

### EXAMPLES

### Stent Preparation

### Example A

In one configuration the invention includes an expandable prosthesis (stent) with a base metal structure comprised of an alloy of nickel titanium which is then encapsulated with a biocompatible porous mesh. The prosthesis self-expands at human or animal body temperature to its full diameter without the aid of a deployment balloon. The mesh pore diameter under scanning electron microscope inspection measures from one micron to five microns. The tubular prosthesis is captured between two sheets of porous PFTE or polyurethane material which have been previously cast by electrospray technique or other means onto a flat surface and are then cut to size before application to the metallic bare stent. A coating comprised of the antiproliferative drug CRC-015 is dissolved into acetone and applied to the stent using a precision syringe applicator which determines the amount of drug applied. The porous mesh covering is capable of storing higher drug doses that are prerequisite to cancer therapy. The coated device is then dried at room temperature for 24 hours. When placed into an appropriate liquid elution media the drug will partition from the mesh surface into the surrounding media. An example of the encapsulated device measuring 40 mm in length and 7 mm in diameter was coated with 4.5 mg of the drug CRC-015 dissolved in acetone solvent. After drying, the coated device was placed into a glass container with 10 mL of 18% acetonitrile in phosphate buffer (w/w). The container was then placed into a Heidoph model Promax 1020 shaker table with a controlled temperature of 37° C. and was shaken at a frequency of 200 strokes per minute. Samples of the liquid media were assayed for drug content. The composite curve of drug elution from the stent over time yields a consistent value of drug shown to release from the stent for a period of at least 60 days.

### Example B

In another configuration a stent comprised of cobalt chromium metal mesh was covered with a polyurethane mesh directly applied using electrospray technology. After drying the covered stent is ready for drug application. A 120 kHz Micromist nozzle from Sonotek Inc. is used to deposit a coating of between 10 and 35 microns depth of drug formulation containing CRC-015 is onto the outer mesh surface. After drying for 24 hours at room temperature the drug coated prosthesis is mounted onto a balloon catheter using a crimping device. In human use when the balloon is located at the site of a stricture or stenosis and inflated the device will deploy to its final diameter and the drug contained in the mesh covering may elute into surrounding tissue.

### Example C

A polymer free solution of CRC-015 was prepared by dissolving the drug in acetone to prepare a solution of 30 mg/mL CRC-015. Next, paclitaxel was added to the solution and dissolved resulting in a final paclitaxel concentration of 10 mg/mL. This drug combined solution was used to coat covered stents by methods disclosed to prepare a therapeutic combination device capable of inhibiting two different metabolic processes of cancer. It is recognized that two drugs can be applied separately to the covered stents resulting with different drugs in different areas or together.

### Example D

A polymer containing solution of CRC-015 was prepared by dissolving CRC-015 in acetone to prepare a solution of 20 mg/mL CRC-015. PLA was added to the solution and dissolved resulting in a final PLA concentration of 10 mg/mL. This drug polymer combined solution was used to coat covered stents by methods disclosed above. The addition of polymers can be utilized to further modify drug elution rates from the covered stent of this invention.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

### Trial A

A study was conducted to evaluate the anti stenotic properties of CRC-015 on a covered self expanding stent as suggested herein implanted in the Superficial Femoral Artery (SFA) and the Profundus Artery in farm swine for 28 days. Two farm swine (species Sus scrofa, ˜3 month of age) were sedated, intubated and placed on isoflurane for general anesthesia. All animals were pre-treated with aspirin (80 mg) and Plavix™ for two days prior to their scheduled surgery and continued daily until their follow up procedure. Two covered stents with a dose of 2.0 μg/mm² CRC-015 having a diameter of 6.0 and 7.0 mm in diameter and a length of 40 mm were deployed in the peripheral SFA and Profundus vessels, for one hind limb. The contralateral femoral artery was used for catheter and guide access. The stent graft material was 2×2 Bioweb Electrospun PTFE and there was no excipient. The electronspun graft material was pressurized at elevated temperature to encapsulate the stent as suggested herein. The delivery system was an over the wire balloon and requires a 0.035″ wire via a dual lumen shaft. The shaft includes a hydrophilic surface coating. The overall length of the delivery system catheter was 150 cm. At necropsy, animals were heparinized then sodium pentobarbital were used for euthanasia. The vascular system was accessed and a sheath inserted into the arterial system. Normal saline was infused into the arterial system until the venous effluent starts to run clear from the pre-treated vessels then the treated arteries were excised. These vessels were adequately labeled, and fixed in 10% neutral formalin and delivered to a pathologist for tissue processing. In one animal the SFA appeared to be occluded while the profundus was widely patent. In the second animal both SFA and profundus were widely patent.

### Trial B

The primary purpose of this study was to evaluate the feasibility of deploying a CRC-015 DCB (drug coated balloon) and a CRC-015 coated biliary stent graft in biliary ducts of domestic swine. Two farm swines (species Sus scrofa, at least 3 month of age) were chosen, because they offer a similar scale as human and thus the instrumentation used in human studies could be used. The animals were euthanized 27 days after successful implantation. Endpoints included duct patency and inflammatory response at 28 days. The CRC-015 DCB and CRC 015 stent graft delivery system as suggested herein was inserted through a direct needle puncture in the common bile duct near the duodenum. Puncture sites were sutured after stent deployment and delivery system retrieval. The DCB was deployed in the proximal portion of the bile duct (near the gallbladder) and the stent graft was deployed in the distal portion of the bile duct (far from the gallbladder). The Test Articles (TA) used in this study were: 6 mm×40 mm CRC-015 DCB, a 0.035 guide wire (4 μg/mm²) and a 6 mm×30 mm CRC-015 biliary stent graft system (4 μg/mm²) as well as 7 mm×30 mm CRC-015 biliary stent graft system (4 μg/mm²).

After sacrificing the animals the facility veterinarian concluded that all animals were in good clinical condition and experienced a normal weight gain over the study period (from treatment to final procedures). The clinical pathology showed some iatrogenic excursions from normal ranges as commonly seen in any surgical animal model due to the blood losses, anesthesia, injectable drugs, surgery and other manipulations (eg. reticulocytosis, increase of AST (Aspartate Aminotransferase) or CK (Creatine Kinase)). No abnormal increases of Bilirubin (Total, Direct or Indirect) were reported at sacrifice suggesting that the TA do not interfere in the normal flux of the biliary duct. The two animals had some excursions pointing to an inflammatory status. One animal also had excursions in platelet count and globulins, but mainly an increased fibrinogen at sacrifice in contrast to a normal level pre-implantation. The role played for the TA, as the possible etiology or a contributor for those changes, remains elusive. The Study Pathologist concluded that necropsy results revealed liver adhesions that were interpreted as secondary to the implantation procedures. Histopathologically, the tissue reaction to the CRC-015 Stent Graft was characterized by minimal or mild subacute inflammation in the mucosa, muscularis and/or serosa, and minimal or mild necrosis of the mucosa and minimal or mild neovascularization. These changes were considered to be related to the bile duct implantation of the stent graft. Multifocal hemorrhages, most probably related to the harvesting of the bile duct, were seen in the muscularis and/or serosa of both animals. Epithelialization of the luminal aspect of the CRC-015 Stent Graft was not observed. Fibrin infiltration, fibrosis, and mineralization were not seen in any of the stent-grafted and the proximal non-stented bile duct sections. In conclusion, the necropsy results revealed liver adhesions that were interpreted as secondary to the implantation procedures. Histopathological evaluation revealed presence of minimal or mild inflammation, neovascularization and mucosal necrosis.

1. A drug eluting stent system for use in delivering at least one therapeutic agent to a target, preferably the bile or pancreatic duct, comprising:
(a) a stent capable of radial expansion; and
(b) a coating, wherein the coating is composed of a porous surface covering and at least one therapeutic agent, wherein the at least one therapeutic agent is an antiproliferative agent and wherein the stent is encapsulated by the coating.

2. The drug eluting stent system of claim 1, wherein the at least one therapeutic agent is a macrocyclic triene immunosuppressive compound.

3. The drug eluting stent system of claim 1, wherein the at least one therapeutic agent has the following structure: 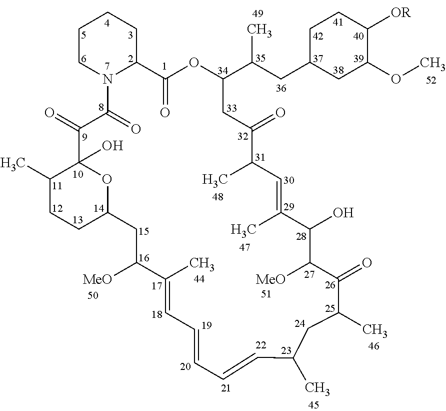
where R is C(O)—(CH₂)ₙ—X, n is 0, 1 or 2, X is a cyclic hydrocarbon having 3-9 carbons and optionally contains one or more unsaturated bonds.

4. The drug eluting stent system of claim 3, further wherein C(O)—(CH₂)ₙ—X has one of the following structures:

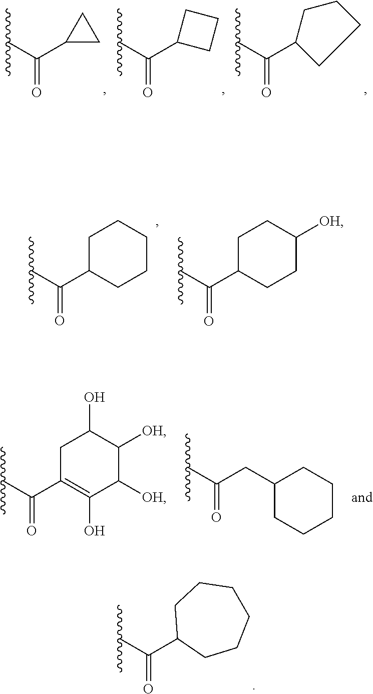

5. The drug eluting stent system of claim 1, wherein the porous surface covering is a nonwoven fabric.

6. The drug eluting stent system of claim 5, wherein the nonwoven fabric is composed of electrospun fibers.

7. The drug eluting stent system of claim 6, wherein the electrospun fibers are composed of a polymer selected from the group comprising or consisting of include polytetrafluoroethylene, fluorinated ethylene propylene, Dacron, polyethylene terephthalate, polyurethanes, polycarbonate, polypropylene, Pebax, polyethylene and biological polymers such as collagen, fibrin, and elastin.

8. The drug eluting stent system of claim 1, wherein the porous surface covering has a porosity of within the range of 20% to 40% of the surface area.

9. The drug eluting stent system of claim 1, wherein the porous surface covering has a porosity of within the range of 20% to 40% of the surface area at the abluminal surface and of less than 5% of the surface area at the luminal surface.

10. The drug eluting stent system of claim 1, wherein the at least the at least one therapeutic agent is present in the porous surface covering in a concentration of greater than 3.0 μg/mm².

11. A method for preparing a drug eluting stent system comprising the steps of
(a) preparing at least one sheet of a porous material on a surface,
(b) mounting the at least one sheet on at least the luminal or abluminal surface of a stent,
(c) applying elevated temperature and pressure to encapsulated the stent in the porous material thereby providing a porous surface covering on the stent,
(d) applying a solution of a therapeutic agent to the porous surface covering, and
(e) drying the porous surface covering applied with the therapeutic agent solution.

12. The method of claim 11, wherein in step (c) the temperature is in the range of 55° C. to 90° C. and the pressure is in the range of 0.13 MPa to 1.25 MPa.

13. The method of claim 11, wherein in step (b) the sheet is mounted on the luminal and abluminal surface of a stent.

14. The method of claim 11, wherein steps (a) and (b) are conducted simultaneously by applying the sheet of porous material directly on the surface of the stent.

15. A method of treating (i) bile duct obstruction or (ii) pancreatic duct obstruction or (iii) pancreatic cancer by placing a drug eluting stent system of claim 1 into the bile duct or the pancreatic duct.
